(19) United States
(12) Patent Application Publication
(10) Pub. No.: US 2014/0148495 A1
(43) Pub. Date: May 29, 2014

(54) MONOLITHIC DRUG DELIVERY SYSTEM

(71) Applicant: University of the Witwatersrand, Johannesburg, Johannesburg (ZA)

(72) Inventors: Sibongile Ruth Sibambo, Komatipoort (ZA); Viness Pillay, Benmore (ZA); Yahya Essop Choonara, Lenasia Ext 1 (ZA)

(73) Assignee: University of the Witwatersrand, Johannesburg, Johannesburg (ZA)

(21) Appl. No.: 14/057,786

(22) Filed: Oct. 18, 2013

Related U.S. Application Data
(63) Continuation-in-part of application No. 12/528,179, filed on Nov. 23, 2009, now abandoned.

(30) Foreign Application Priority Data
Feb. 23, 2007 (ZA) 2006/09747

(51) Int. Cl. A61K 9/00

(52) U.S. Cl. CPC A61K 9/0087
USPC 514/415; 514/772

(57) ABSTRACT

An improved monolithic drug delivery dosage form releases a pharmaceutically active agent at a predetermined rate. The dosage form comprises a salted-out or crosslinked polymer and a pharmaceutically active agent. The salted-out or crosslinked polymer functions to polymerically entangle the pharmaceutically active agent but, progressively relaxes on contact with an aqueous medium in use to release the pharmaceutically active agent at a predetermined rate.

### CROSS-REFERENCE TO RELATED APPLICATIONS

This application is a continuation-in-part of U.S. patent application Ser. No. 12/528,179 filed Nov. 23, 2009, which is a National Stage filing of International Patent Application No. PCT/IB2008/000396 filed Feb. 22, 2008, which claims the benefit of South African Patent Application No. 2006/09747 filed Feb. 23, 2007. The contents of the aforementioned applications are hereby incorporated by reference in their entirety.

### FIELD OF THE INVENTION

The field of this invention is the application of salting-out and crosslinking of polymers, preferably polyesters to modify the physicochemical and physicomechanical properties of the said polymers and producing a rate modulated drug delivery system.

### BACKGROUND

The correlation between the physicochemical and physicomechanical modifications of polymeric materials as well as the related release kinetics from drug delivery devices is significant to our understanding and elucidation of the mechanisms by which phenomena such as salting-out and crosslinking occur (Dashevsky, et al., 2005; Dayal et al., 2005; Huang et al., 2005; Jones et al., 2005; Young et al., 2005).

Salting-out and cross-linking have major implications on the transitions of the physicochemical and physicomechanical properties of polymers that impact on the release kinetics of drug delivery devices and phenomena such as diffusion, relaxation and erosion. (Avgoustakis, 2004; Izutsu and Aoyagi, 2005). The alteration of the three-dimensional polymeric network that results from changes in bond vibrations, morphology, resilience and glass-transition temperature can be attained through ionic interactions between polymer-salt and polymer-polymer during salting-out and crosslinking (Cao et al., 2006).

Salting-out, a colloidal phenomenon, has the capacity to change the morphology, resilience and glass-transition temperature of polymers, by means of salts that cause stochastic fluctuations of the free energy proportional to the salt concentration (Horvath, 1985; Tanaka and Takahashi, 2000). Zhang et al. (1995) showed that the salts can also modulate the release and swelling kinetics of bioerodible polyesters. Pillay and Fassihi (1999) reported on how electrolyte inclusions can alter the configuration and the micro-environment within hydrating matrices to control their swelling kinetics as well as physical rigidity. Furthermore, Hiroshu (2003) described the complexation of divalent ions such as calcium and magnesium to polyesters by ion-dipole bonds. The presence of these ionizable salts allows for non-collapsible diffusion channels to form within the polymeric structure. As the matrix hydrates, the salts and polymer compete for water of hydration, resulting in a programmed release rate (Pillay and Fassihi, 2001, Swenson 2001). Thus the salts will attract water molecules in an effort to solvate themselves, thereby dehydrating the polymer.

One of the principal mechanisms of salting-out is the salt-induced surface tension increase of the water molecules (Eigen and Wicke, 1964; Meander and Horvath, 1977). Electron donor/acceptor interactions are a significant part of the salting-out technique, since the various anionic and cationic species in aqueous solution order certain extents of changes according to their efficacies in salting-out thermoplastic polymers such as OH polyester. Thermodynamic studies by Arakawa and Timashef (1982) demonstrated that the salts that decrease dissolution of hydrophobic polymers are preferentially excluded from the vicinity, strongly bind to the polymers and are called kosmotropes, whereas salts that increase the polymer solubility display weak preferential binding with the polymer and tend to settle at the polymeric surfaces (Galinski et al., 1997; Moelbert et al., 2004).

Although there is a large variety of forces present, including electrostatic and Lifshitz-Van der Waals, the interactions responsible for the salting-out phenomena seem to be dominated by the hydration forces ruled by electron donor/acceptor. Kosmotropes tend to tighten the inter- and intra-molecular structure allowing polymeric interactions, thus enhancing the polymeric properties that include resilience, energy of absorption and the deformability modulus of the polymer.

In aqueous polymeric solutions, salting-out creates stabilization of the water structure, thereby decreasing the hydrogen-bonding between water molecules and the polymeric chain. This is an alternate mode that enhances the hydrophobic interaction between polymeric chains (Bolen and Baskakov, 2001; Valery et al., 2004). These chains are rendered stiff by the introduction of chemical bonds between their monomers (crosslinking), and between the polymeric chains and the salts, which further transform the properties of the polymer (Nystrom et al., 1995). The resulting crosslinked polymeric networks are dimensionally stable, with minimal hydrolysis of the polymer bonds, and exhibit superior structural integrity, making them suitable for sustained drug delivery applications. While polymeric strengths are controlled by the degree of crosslinking, the degradation rate of these networks can be controlled independently by the chemical composition.

Furthermore, in addition to the potential transitions of the polymeric properties, the drug release kinetics and mechanisms may also be significantly influenced by a change in the physicomechanical properties of the polymeric material. Various studies have reported on the mechanisms of drug release from monolithic polymeric devices. In principle, a monolithic device is a simple drug delivery system comprising homogenous drug dispersed within a polymeric matrix. Langer and Peppas (1981) proposed that during the overall release of drugs from monolithic matrices, two distinctive processes could be observed, namely, swelling and ‘true’ dissolution of the polymer. In case of a swellable system, the device will immediately swell once in contact with the dissolution media. Thus the drug release is controlled by the hydration rate of the system. In order to minimize an initial rapid drug release phase, the polymer employed must be able to form a ‘protective’ gel layer prior to dissolution. Designing a monolithic system for providing controlled drug release kinetics for water-soluble drugs such as, melatonin, is often a challenge. Pillay and Fassihi, 2000, postulated that these drawbacks may be attributed to the following factors, such as:
(i) The increased hydrophilicity of the drug that causes a burst effect during drug release;
(ii) The lack of accurate management of polymer relaxation or disentanglement over time-dependent processes in relation to drug dissolution and diffusion; and
(iii) The complexity of controlling the increase in the diffusional pathlength with time is not easily attainable (Pillay and Fassihi).

The inherent ineffectiveness of this system can however, be manipulated through the use of salts to modulate the internal geometry of the system. Salts have been highly successful in controlling dissolution and drug release, by demonstrating differential swelling boundaries and texturally variable matrices that manifest as ‘peripheral matrix stiffening’, a phenomenon that retards the release of water-soluble drugs. Therefore, in this work, we evaluated the physicochemical and physicomechanical transitions occurring within salted-out polylactic-co-glycolic acid (PLGA), an α-OH polyester, using a statistical approach to develop a mechanistic understanding of its ability to control the release of melatonin from a monolithic drug delivery matrix. These salted-out and/or crosslinked complexes were termed ‘PLGA scaffolds’.

### SUMMARY OF THE INVENTION

In accordance with this invention there is provided a monolithic drug delivery dosage form comprising a salted-out or crosslinked polymer and a pharmaceutically active agent disposed therewith, the salted-out or crosslinked polymer functioning to polymerically entangle the pharmaceutically active agent but, progressively relaxes on contact with an aqueous medium in use to release the pharmaceutically active agent at a predetermined rate.

There is also provided for the salted-out or crosslinked polymeric material to be poly-lactic co-glycolic acid that is able to control, in use, the release of a pharmaceutically active agent over a prolonged period of time depending on the rate of polymeric relaxation of the polymer on exposure to an aqueous medium.

There is also provided for the salting-out or crosslinking reaction to occur either in combination with the pharmaceutically active agent alternatively with the polymeric material on its own to cause stochastic fluctuations of the reaction which results in polymeric entanglement of the pharmaceutically active agent.

There is further provided for polymeric relaxation reaction to occur in a time dependent manner from the outer boundaries of the dosage form towards its inner boundaries and thus limit outward diffusion of the entangled pharmaceutically active agent in a controlled fashion as the inward ingress of aqueous medium causes a progressive relaxation of the polymeric chains from the outer boundaries of the dosage form or tablet in a direction towards its inner core.

There is also provided for the sating-out and crosslinking reactions of the polymer to include use of a crosslinking reagent, preferably an inorganic salt further preferably an inorganic ionic salt, preferably from the Hofmeister series of salts examples of which are sodium chloride, aluminium chloride and calcium chloride.

There is further provided for the polymer to be a polyester, preferably a poly-lactic acid and/or its co-polymers and further preferably poly-lactic co-glycolic acid.

The invention extends to a method of producing a monolithic drug delivery dosage form comprising a salted-out or crosslinked polymer and a pharmaceutically active agent disposed therewith comprising salting-out or crosslinking a polymer to polymerically entangle the pharmaceutically active agent but, progressively relaxing on contact with an aqueous medium in use to release the pharmaceutically active agent at a predetermined rate.

There is also provided for the salted-out or crosslinked polymeric material to be poly-lactic co-glycolic acid that is able to control, in use, the release of a pharmaceutically active agent over a prolonged period of time depending on the rate of polymeric relaxation of the polymer on exposure to an aqueous medium.

There is also provided for the salting-out or crosslinking reaction to occur either in combination with the pharmaceutically active agent alternatively with the polymeric material on its own to cause stochastic fluctuations of the reaction which results in polymeric entanglement of the pharmaceutically active agent.

There is also provided for the salting-out and crosslinking reaction of the polymer and a crosslinking reagent, preferably an inorganic salt further preferably an inorganic ionic salt, preferably from the Hofmeister series of salts examples of which are sodium chloride, aluminium chloride and calcium chloride.

There is further provided for the polymer to be a polyester, preferably a poly-lactic acid and/or its co-polymers and further preferably poly-lactic co-glycolic acid.

In a preferred embodiment of this invention, there is provided a monolithic drug delivery dosage form comprising a salted-out and crosslinked polymer having a pharmaceutically active agent disposed therewith, wherein the polymer is poly-lactic co-glycolic acid and is crosslinked and salted-out with a crosslinking agent selected from the group consisting of: sodium chloride, aluminium chloride and calcium chloride, such that bonding occurs between the polymer and the crosslinking agent to form an independent crosslinked and salted-out product which entangles the pharmaceutically active agent, wherein the monolithic drug delivery dosage form has a zero order release of the pharmaceutically active agent on contact with an aqueous medium.

The monolithic drug delivery dosage form may be compressed forming a tablet, typically compression takes place through aid of a hydraulic press.

The poly-lactic co-glycolic acid may have a 1:1 lactide:glycolide ratio.

The zero order release of the pharmaceutically active agent may last for a period of up to 30 days.

Preferably, the pharmaceutically active agent may be water soluble, further preferably the pharmaceutically active agent may be melatonin.

The invention extends to a method of producing the monolithic drug delivery dosage form. In another preferred embodiment of this invention there is provided for a method of producing a monolithic drug delivery dosage form comprising a pharmaceutically active agent characterised in that the method includes the steps of salting-out and crosslinking poly-lactic co-glycolic acid with a crosslinking agent selected from the group consisting of: sodium chloride, aluminium chloride and calcium chloride.

The steps of salting-out and crosslinking poly-lactic co-glycolic acid with a crosslinking agent may typically include the follow steps:
(a) dissolving poly-lactic co-glycolic acid in a water miscible solvent to form a polymeric solution;
(b) adding the pharmaceutically active agent to the polymeric solution;
(c) adding a crosslinking agent selected from the group consisting of: sodium chloride, aluminium chloride and calcium chloride, so as to entangle the pharmaceutically active agent with the poly-lactic co-glycolic acid; and
(d). salting-out the crosslinked poly-lactic co-glycolic acid of step (c).

The poly-lactic co-glycolic acid may have a 1:1 lactide:glycolide ratio.

The water miscible solvent may be an organic solvent being at least one selected from the group comprising: acetone and N,N-dimethyl formamide (DMF).
Step (d) may include the addition of H3O+ to facilitate salting-out.
The method may further comprise an additional step, Step (e) comprising compressing the crosslinked and salted-out poly-lactic co-glycolic acid to form a tablet.

The pharmaceutically active agent may be water-soluble, preferably the pharmaceutically active agent may be melatonin.

### BRIEF DESCRIPTION OF THE DRAWINGS

Embodiments of a monolithic drug delivery dosage form according to the invention will be described below with reference to the accompanying figures in which:

FIG. 1 illustrates typical Force-Distance and Force-Time profiles of PLGA scaffolds for determining (a) energy absorbed (b) deformability modulus and (c) matrix resilience (N=10);

FIG. 2 is a series of selected scanning electron micrographs of PLGA scaffolds demonstrating the surface morphology of PLGA: salted-out with NaCl [(a) and (b)], CaCl₂. [(c) and (d)], and AlCl₃ [(e) and (f)];

FIG. 3 shows profiles depicting differences in the physicomechanical properties of PLGA scaffolds. Plot (a) Resilience (R), (b) Energy absorbed (E) and (c) Deformability modulus (DM);

FIG. 4 shows typical surface response plots depicting the effects of the independent formulation variables on the physicomechanical properties of the salted-out PLGA scaffolds, namely (a) Resilience (R) (%); (b) Energy absorbed (E) (J); and (c) Deformability modulus (DM) (N/mm). AC═AlCl₃, CC═CaCl₂, SC═NaCl.−1=0% w/v 0=5% w/v 1=10% w/v;

FIG. 5 presents a series of typical profiles used to construe the main and interactions effects of NaCl (a) and (d); CaCl₂ (b) and (e); AlCl₃ (c) and (f) for resilience in the presence of a combination of parameters;

FIG. 6 illustrates profiles demonstrating the correlation between experimental and fitted response values. R=Resilience; E=Energy absorbed; DM=Deformability modulus;

FIG. 7 shows five superimposed FTIR profiles depicting transitions from native PLGA to salted-out PLGA scaffolds. (a)=Native PLGA, (b)-(e)=PLGA salted-out with NaCl, CaCl₂—AlCl₃ and a combination of NaCl+CaCl₂+AlCl₃ respectively;

FIG. 8 illustrates Differential Scanning calorimetry (DSC) profiles of native and salted-out PLGA scaffolds demonstrating thermal transitions of (a) native PLGA, (b) PLGA salted-out with NaCl, (c) CaCl₂, (d) AlCl₃, and (e) a combination of NaCl, CaCl₂, and AlCl₃;

FIG. 9 shows release profiles of melatonin from polymer when drug was either (a)(b) non-crosslinked or (c) crosslinked during the formation of PLGA scaffolds;

FIG. 10 shows a salted-out and cross-linked scaffold depicting the exterior surface morphology;

FIG. 11 shows PLGA interactions where (a) illustrates PLGA, an α-OH polyester, interacting with DMF (b) illustrates interaction with water to solubilize PLGA (note outer solvation shell depicting water molecules), and (c) illustrates interaction after H3O+ addition (note the temporary charge transfer from water molecules to DMF molecules); and

FIG. 12 shows response surface plots depicting matrix resilience (MR) vs. (a) water volume and PLGA concentrations, (b) PLGA molecular mass and PLGA concentration, (c) water volume and PLGA concentration, (f) PLGA molecular mass and PLGA concentration.

### DETAILED DESCRIPTION

### Example A

### 1. Materials and Methods

### 1.1 Materials

PLGA was obtained from Boehringer Ingelheim Pharma (Ingelheim, Germany) (Resomer® RG504 50:50 lactide:glycolide; M_{w} 48,000; i.v. 0.48-0.60 dl/g). A water miscible solvent, typically a water miscible organic solvent, was used, in this case acetone was used as a solvent, and analytical grades of sodium chloride (NaCl), calcium chloride (CaCl₂), (Rochelle Chemicals, South Africa) and aluminium chloride (AlCl₃) (Merck, Darmstadt, Germany) were used as the ionic salts. Disodium hydrogen orthophosphate, (Na₂HPO₄), and potassium dihydrogen phosphate (KH₂PO₄) were obtained from Saarchem (Pty) Ltd., South Africa, Model drug, melatonin was obtained from Sigma-Aldrich Co., Germany.

### 1.2 Building the Experimental Design

A 3 factor, 3 level Box-Behnken statistical design was built in order to model the number of experiments needed for formulation optimisation and to establish the main and interaction effects of the independent formulation variables on the physicochemical and physicomechanical properties of the PLGA scaffolds using Minitab V14 (Minitab, USA).

### 1.3 Preparation of PLGA Scaffolds

PLGA scaffolds were prepared by salting-out and subsequent crosslinking using a combination of acetone and ionic salts in accordance with a Box-Behnken design template outlined in Table 1. Fourteen polymeric solutions comprising 0.4 g of PLGA dissolved in 15 mL of acetone were prepared. The crosslinking solutions comprising 75 mL of 0 w/v, 5 w/v or 10% w/v of NaCl, CaCl or AlCl₃ was added to the polymeric solution and agitated fir a period of 30 minutes. The resultant PLGA scaffolds were removed from the crosslinking solution washed thrice with 500 mL deionized water and dried to constant mass at room temperature. Each PLGA scaffold was stored for a maximum of 48 hours prior to physicochemical and physicomechanical evaluation.

TABLE 1
Box-Behnken design template with randomly
generated PLGA Scaffold formulations

| | Randomized Run | [NaCl] | [CaCl₂] | [AlCl₃] |
|---|---|---|---|---|
| Formulations | Order | (% ^{w}/ᵥ) | (% ^{w}/ᵥ) | (% ^{w}/ᵥ) |
| 1 | 6 | 10 | 5 | 5 |
| 2 | 11 | 5 | 10 | 0 |
| 3 | 7 | 5 | 5 | 5 |
| 4 | 4 | 5 | 0 | 10 |
| 5 | 2 | 10 | 5 | 10 |
| 6 | 3 | 5 | 10 | 0 |
| 7 | 5 | 5 | 5 | 5 |
| 8 | 12 | 10 | 5 | 10 |
| 9 | 8 | 5 | 10 | 0 |
| 10 | 13 | 5 | 5 | 5 |
| 11 | 9 | 5 | 0 | 10 |
| 12 | 14 | 10 | 5 | 10 |
| 13 | 1 | 5 | 10 | 0 |
| 14 | 10 | 5 | 5 | 5 |

The quadratic model for the responses is shown in Equation 1:

Response=b₀+b₁[NaCl]+b₂[CaCl₂]+b₃[AlCl₃]+b₄[NaCl]²+b₅[NaCl][CaCl₂]+b₆[NaCl][AlCl₃]+b₇[CaCl₂][AlCl₃]+b₈[CaCl₂]²+b₉[AlCl₃]² (Equation 1)

Where, the Response is associated with each factor level, b₀ . . . b₉ are the regression coefficients, and [NaCl], [CaCl₂] and [AlCl₃] are the independent formulation variables.

### 1.4 Morphological Characterization of the PLGA Scaffolds

The surface morphology of the PLGA scaffolds was assessed from Scanning Electronic Microscopic (SEM) images employing a thermal emission JEOL, JSM-(Japanese Electronic Optical Laboratories, Tokyo, Japan) electron micrograph. Samples of PLGA scaffolds were sectioned and mounted on aluminium stubs prior to sputter-coating with a layer of carbon. Each sample was viewed under varying magnifications at an accelerating voltage of 20 kV.

### 1.5 Determination of the Physicomechanical Properties of the PLGA Scaffolds

The physicomechanical properties of the PLGA scaffolds were evaluated using a Texture Analyzer (TA.XTplus Texture Analyzer, Stable Microsystems, UK). Stress-strain profiles with a high degree of accuracy and reproducibility were capture at a rate of 200 points per second employing Texture Exponent software V3.2 and subsequently analyzed. A 3.5 cm flat-tipped circular steel probe was attached to the force transducer. The parameter settings employed to obtain the energy absorbed, deformability modulus and matrix resilience are shown in Table 2.

TABLE 2
Textural parameter settings

| | Energy absorbed and | | | |
|---|---|---|---|---|
| Settings | Deformability modulus | | Matrix resilience | |
| Pre-test speed | 1 | mm/sec | 1 | mm/sec |
| Test speed | 0.5 | mm/sec | 0.5 | mm/sec |
| Post-test speed | 1 | mm/sec | 1 | mm/sec |
| Compression force/strain | 40N | | 50% | |
| Trigger type | Auto | | Auto | |
| Trigger force | 0.5N | | 0.5N | |
| Load cell | 50 | kg | 50 | kg |
| Distance | 20 | mm | 20 | mm |

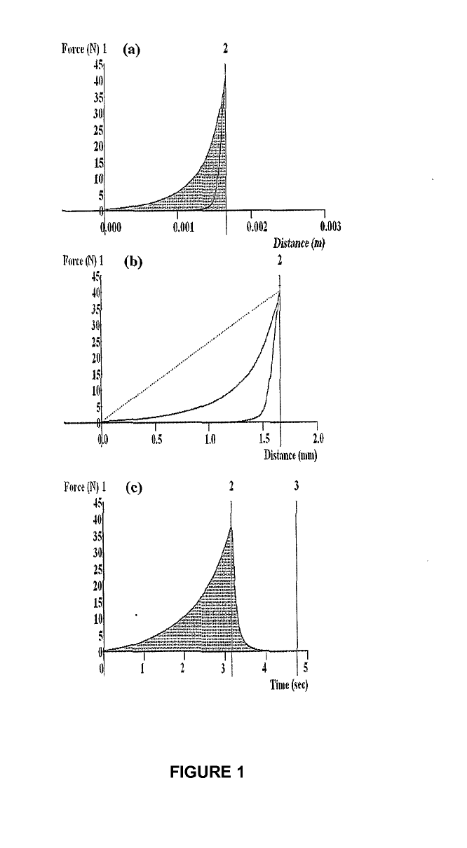

To elucidate the energy absorbed, matrix resilience and deformability modulus, Force-Distance and Force-Time profiles for each PLGA scaffold was generated. Typical textural profiles used for quantifying the physicomechanical properties are depicted in FIG. 1.

FIG. 1(a) depicts the anchors used in a Force-Distance profile for calculating the energy absorbed i.e. the total area under the curve (AUC) [Nm=Joules] between anchors 1 and 2. FIG. 1(b) depicts the anchors employed for determining the deformability modulus (the tendency of the PLGA scaffolds to change shape upon the application of stress) i.e. the gradient between anchor 1 and the maximum force attained during sample analysis. FIG. 1(c) depicts the anchors employed for calculating the matrix resilience, i.e. the ratio of the AUC between anchors 2 and 3 and 1 and 2 (AUC₃₂/AUC₁₂) for a Force-Time profile. Note that the resilience may be defined as

1.6 Elucidation of the Molecular Structural Transformations within PLGA Scaffolds

Fourier Transform Infra-Red (FTIR) spectroscopy was performed on native PLGA and PLGA scaffolds to determine chemical transformations potentially occurring within the polymeric backbone due to salting-out and subsequent crosslinking using a Nicolet Impact 400D (Nicolet Instrument Corporation, Pennsylvania, USA) instrument. The potassium bromide (KBr) disc approach was employed, whereby 7.5 mg samples of each PLGA scaffold was triturated with 200 mg of KBr and compressed in a transparent circular disc using a Beckman Hydraulic Press (WIKA Instruments (Pty) Ltd, Johannesburg, South Africa). Background scans were obtained for all samples and the % transmittance was recorded between 1000-400 cm⁻¹ at an intermediate resolution.

### 1.7 Thermal Transition Analysis of the PLGA Scaffolds

Differential Scanning calorimetry (DSC) was used to record transitions in specific heat capacity and latent heat, which indicated changes in the amorphous or crystalline structure as a result of scaffold formation from crosslinking native PLGA. Thermal transitions were recorded on a Perkin-Elmer Pyris-1 connected to a controller model TAC1/DX (Perkin-Elmer, Inc, USA). Samples were heated in increments from 25° C. to 400° C. at a rate of 10° C./min. Samples of 5-10 mg of each PLGA scaffold was placed within a crimped aluminium pan and subjected to the heat gradient. Thermograms were obtained and subsequently analyzed.

### 1.8 Preparation of the Salted-Out PLGA Monolithic Matrix

Formulations of either drug-free PLGA or drug-loaded PLGA samples were salted-out at various concentrations in accordance with a Box-Behnken statistical design. Matrices were prepared by direct compression of a mixture comprising 300 mg salted-out PLGA and 10 mg melatonin for the drug-free PLGA and 350 mg of each drug-loaded variant was compressed using a Beckman Hydraulic Press (Beckman Instruments, Inc., Fullerton, USA). Therefore, the PGLA scaffolds were compressed to form tablets.

### 1.9 Drug Entrapment Efficiency (DEE) of the PLGA Scaffolds

DEE studies were performed by immersing each scaffold in 100 mL acetone to effect complete dissolution of the scaffold. Thereafter, melatonin content was established in triplicate using UV-spectroscopy at 278 nm.

1.10 In Vitro Drug Release from the Monolithic Matrices

Drug release studies were conducted in 500 mL phosphate buffered saline (PBS) (pH 7.4; 37° C.) using a modified USP25 apparatus at 50 rpm. Melatonin assays were performed with UV-spectroscopy (278 nm) (SPECORD 40, Jena, Germany). The dissolution data was subjected to a model-independent analysis known as the time-point approach. Briefly, the mean dissolution time set at 30 days (MDT₃₀) for each formulation was calculated. The application of the mean dissolution time provided a more precise analysis of the drug release performance and a more accurate comparison of several dissolution data sets. Equation 2 was employed in this regard:

$$M D T = {\sum }_{i = 1}^{n} ti \frac{Mt}{M ∞}$$

(Equation 2)

Where Mₜ is the fraction of dose released in time tᵢ=(tᵢ+tᵢ₋₁)/2 and M_{∞} corresponds to the loading dose.

### 2. Results and Discussion

### 2.1 Proposed Interactions Between Native PLGA Polymeric Chains and Crosslinking Ions

The solvated ion pairs of Na⁺, Ca²⁺, and Al³⁺ develop into electron nodes that facilitate ionic reactions. These solvated ion pairs are able to attract the adjacent cations of Cl⁻ and O²⁻ within the polymeric matrix thus contributing to crosslinking of lactide and glycolide chains within the PLGA molecular structure. This crosslinking reaction depends primarily on the ionization energies of the salting-out ion, hydration enthalpies in solution and the thermodynamic stability of the monomeric PLGA units. Furthermore, the coordination number of each salt, 8, 6 and 4 for Al³⁺, Na⁺ and Ca²⁺ respectively and atomic size of ions (Table 3) also influences the attraction of adjacent cations during crosslinking with the ion and/or salt possessing the highest coordination number and atomic size having the most influence on the crosslinking reaction and subsequently contributes a central factor in modifying the native PLGA polymeric structure to produce a robust PLGA scaffold.

TABLE 3
Physicochemical properties of the salts employed
during crosslinking of native PLGA

| | Atomic radius of | Metal Coordination | Cation Coordination |
|---|---|---|---|
| Salt Type | Metals (pm) | Number | Number |
| NaCl | 186 | 6 | 6 |
| CaCl₂ | 197 | 4.2 +/− 0.5 | 5.4 +/− 0.3 |
| AlCl₃ | 125 | 8 | 6 |

The salts used in this study, namely NaCl, CaCl₂ and AlCl₃ differ with regard to the physicochemical, physicomechanical and morphological structure of the resultant PLGA scaffolds. Furthermore, the shape and stereo-orientation within the PLGA structure differs and therefore, the ability of water molecules to be imbibed within the matrix depends on the rate of polymer-salt interactions, the reactivity, atomic size and coordination number of the concerned ions, the nature of the crystal-lattice packing of ions and the polymeric substrate present during salting-out and subsequent crosslinking.

### Scanning Electron Microscopic Image Analysis of the PLGA Scaffold Morphology

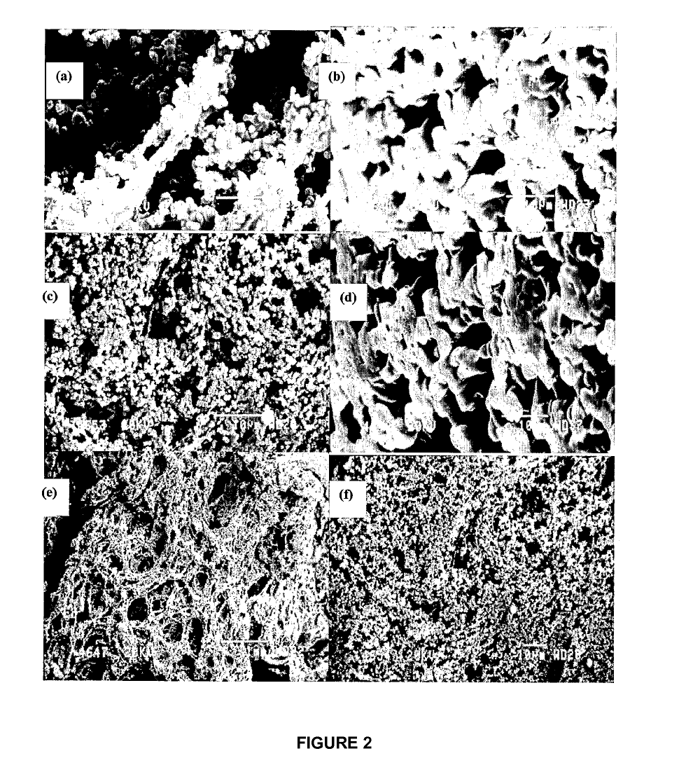

Three-dimensional architecture comprising various fiber volume and diameters, interconnections and pore sizes were obtained from polymer-salt interactions as a result of crosslinking during PLGA scaffold formation. The presence of salts generated areas of high entropy at the solid-liquid interfaces resulting in altered fibrous structures with distinct morphologies (FIG. 2). The ionic interactions between the salts and PLGA molecules were dependent on the ionization energies of crosslinking ions, hydration enthalpy in solution as well as the thermodynamic stability and molecular accumulation of salts and water at the lactide-glycolide strands of native PLGA. As a result several morphological conformations of each PLGA scaffold were obtained (FIG. 2). Furthermore, the coordination number of each salt influenced the number of covalent bonds formed. Hence, hydrogen bonding and other intra and inter-ionic forces located on the PLGA molecule (oxygen residues) cluster-packed with water molecules decided the nature and size of pores and fibers of the newly formed PLGA scaffolds.

SEM images of PLGA scaffolds salted-out with NaCl revealed a uniform distribution of interconnected pores, divided by struts of microporous structures that maintained homogeneity of crosslinked fibers in a neuronal meshwork archetype (FIGS. 2 a and 2 b). These fibers possessed pores and fiber diameters ranging between 0.1-1.4 μm, and fiber volumes ranging from 0.01-0.03 μm³. In FIGS. 2 c and 2 d, the divalent salt CaCl₂ produced a fibrillar composite PLGA scaffold with interconnecting channels in a distinct voluminous trabecular formation with scales of pore sizes and diameters ranging between 7.5-15 μm and fiber volumes of 800-14000 μm³. In FIGS. 2 e and 2 f, PLGA scaffolds salted-out with a trivalent salt namely AlCl₃ revealed fine fibrous morphologies with distinct crosslinks that resulted in a ramified interconnection of the PLGA scaffold design with pore sizes and diameters ranging from 0.03-0.10 μm and fiber volumes between 0.09-0.17 μm³.

In general, introduction of salting-out ions to the polymeric solution facilitated the native PLGA polymeric structure to assume a fixed three-dimensional configuration into crosslinked lactide-glycolide chains. Furthermore, the adjacent voids resulting from such a configuration were able to accommodate water molecules in accordance to the dimensions of voids created due to short and long distance interactions between native PLGA chains. These voids significantly provided the space for binding water molecules which was dependent on the rate of the salting-out and subsequent crosslinking reaction. An increase in the fibrillar nature of the PLGA scaffolds augmented the physicomechanical properties such as matrix resilience. The distinct differences in morphology revealed in each micrograph of the PLGA scaffolds suggest the versatility of the scaffold and hence may be suitable for tailored manufacturing that match specific applications.

### 2.3 Textural Profile Analysis to Quantify the Physicomechanical Properties of PLGA Scaffolds

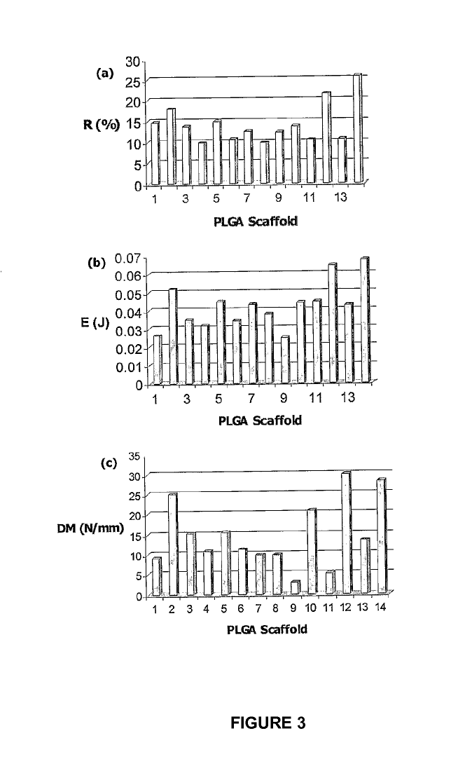

Analysis of the textural profiles provided an insight on the Stress-Strain relationships for the PLGA scaffolds. Results demonstrated that native PLGA could be modified into a highly resilient polymeric material by rapid ionic salting-out and subsequent crosslinking in order to achieve elasticity that depended mainly on the type and the concentration of salt employed. The matrix resilience values of PLGA scaffolds salted-out with NaCl and AlCl₃ were superior to that of PLGA scaffolds salted-out with CaCl₂ (FIG. 3).

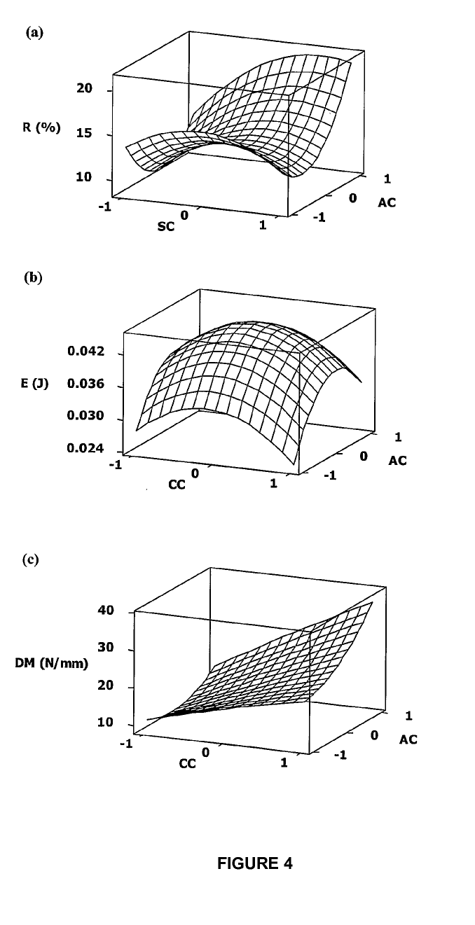

The physicochemical nature of the salt employed during salting-out created a micro-environment which accentuated the viscoelastic behaviour of the PLGA scaffold. The viscoelasticity caused densification of the PLGA scaffolds, which resulted in resistance of the scaffold to deform under stress during textural analysis. FIGS. 4 a and 4 b revealed significant increases in the resilience and energy absorbed when concentrations of NaCl and AlCl₃ were increased, for instance in formulations 2, 12 and 14. The concentration of CaCl₂ was found to be inversely proportional to the energy absorbed as demonstrated in formulations 6, 9 and 13. The increase in resilience and deformability moduli was linearly correlated to the quantity of energy absorbed by the PLGA scaffolds per unit volume shown in FIGS. 4 b and c.

The modification of the physicomechanical properties revealed by these profiles is consistent with the dense surface morphology of the PLGA scaffolds depicted in FIG. 2. In general, salts that produced more compact polymeric structures with smaller and more uniform pores, such as NaCl and AlCl₃ accentuated the physicomechanical properties of the PLGA scaffolds, whereas salts that produced larger pores decreased the resilience.

Furthermore, the resultant accumulation of additional water molecules conferred more dipoles to the matrix, which were less responsive to activity within the PLGA backbone. Ferry (1980) reported that the presence of water molecules which are dipole and contributors of interfacial tension decreases matrix resilience. The proximity of the polarising dipoles to the PLGA backbone and the extent to which they are influenced by the configurational activity of the PLGA chain directly influenced the total resilience, energy absorbed, and deformability modulus of the PLGA scaffold.

### 2.4 Response Surface Plots Indicating Interaction Between Dependent Variables

Surface plots (FIG. 4) were constructed to visually demonstrate the individual and synergistic effects of the salts on modifying the physicomechanical properties of native PLGA by scaffold formation. FIG. 4 a revealed that at lower concentrations (between 0-5%%), NaCl significantly increased resilience of PLGA scaffolds up to a limit of 15% at which any further increase of NaCl (above 5% w/v) resulted in a decreased resilience. At low concentrations (between 0 and 5(w/v) of AlCl₃ a resilience of 10% was maintained and a further increase in AlCl₃ beyond 5% w/v resulted in a linear increase of resilience.

FIG. 4 b demonstrated that a concentration of CaCl₂ above 5% w/v lowered the energy absorbed and that a 10% w/v CaCl₂ significantly diminished the ability of the PLGA scaffolds to absorb energy. Furthermore, FIG. 4 c depicts that the concentration of CaCl₂ had a minor effect on the deformability modulus of the PLGA scaffolds, whereas an increase in AlCl₃ concentrations largely increased the deformability modulus of the PLGA scaffolds. The following three-dimensional surface plots depict each of the responses (physicomechanical properties) resulting from changes in the independent formulation variables.

### 2.5 Determination of the Main and Interactions Effects on the Various Responses

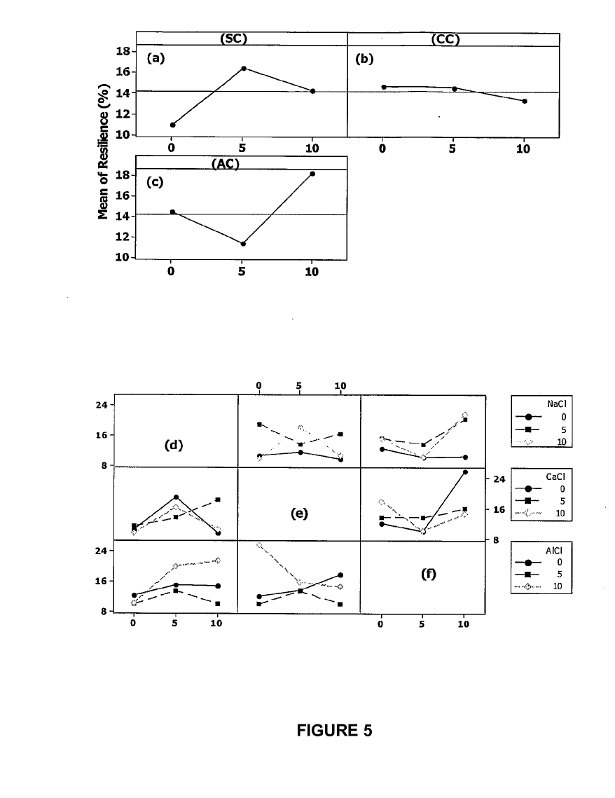

The main and interaction effects of the salt type and concentration and their influence on the physicomechanical properties of PLGA are demonstrated in FIG. 5. The plots of main and interaction effects were run to provide a visual authentication of the significant variables on the resilience, energy absorbed, and deformability modulus model terms. The effects on the responses were found to be attributable to the main effects (i.e. the salts) as well as other interactions such as the polymeric substrate, solvent, water volume, and salting-out reaction time up to the last variable interactions. The degree of interactions was observed to rise exponentially with the number of factors. Digression from the centre-point designated a change in response over the tested range. Visually, the discrepancies in the mean values of the plot are the least squares estimate for the effect. Huge discrepancies indicated by higher gradients as in FIGS. 5 a, c, d, and f signified important variables while diminutive discrepancies indicated by lower gradients signified trivial variables in a given plot. Parallel plots as in FIGS. 5 b and 5 e implied minimal or no interaction of the independent formulation variable.

As demonstrated in FIG. 5, the type and concentration of salt played a vital role in the nature and extent of PLGA modification. In FIG. 5c NaCl, a monovalent salt had the greatest effect on resilience with optimal resilience experienced at 5% w/v. The divalent salt CaCl₂ had a minor effect on resilience, regardless of the change in concentrations. It was also observed that the resilience increased with the increase in concentration of the trivalent salt AlCl₃ from 5% w/v. A similar correlation could be seen from FIG. 3 and FIG. 4. These observed transitions in resilience can be explained as a contribution from a combination of several effects such as variations of the water molecule structure present in the matrix hydration sheath as well as the adjustments of the interactions between the PLGA and solvent due to the presence of various salts.

### 2.6 Correlation Between the Experimental and Fitted Responses Employing a Quadratic Model

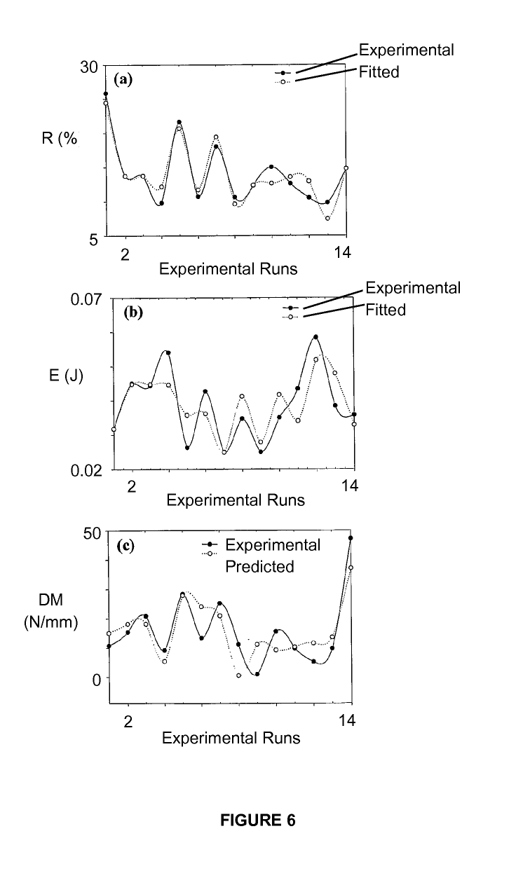

FIG. 6 depicts the close correlation between the fitted and experimental values for the dependent formulation variables, namely, resilience, energy absorbed, and deformability modulus. No significant differences were noted between the fitted and experimental values (p>0.05). This therefore, indicated that the Box-Behnken design provided a suitable statistical approach to evaluate the effects of various salts on modifying native PLGA into salted-out PLGA scaffolds.

### 2.7 Assessment of the Polymer-Salt Interactions and Polymeric Structural Transitions

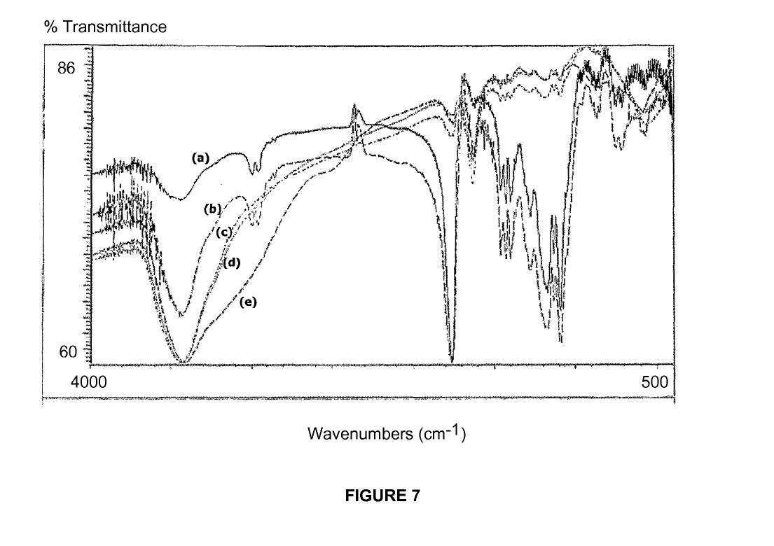

As observed in the FTIR profiles depicted in FIG. 7, the functional groups of PLGA involved in interactions with the salts were similar. However, the degree and extent of these bond vibrations at finger-print regions varied. This implied that the polymer-salt interaction in solution was clearly influenced by the molecular structure of the salt as well as the chemical backbone of PLGA that resulted in the diverse morphological, physicochemical, and physicomechanical transitions demonstrated by the PLGA scaffolds.

During salting-out and subsequent crosslinking the salts ionized in water and reacted with the δ, π, σ, C—O and H-groups, to form hydrogen, ether bonds and salt-oxygen bonds between the PLGA chains. This resulted in crosslinking of the lactide-glycolide units within the PLGA molecular structure, Hydrogen, ether and ion-oxygen bonds were formed by the salts between free pendant carbonyl groups of PLGA into resonance stabilized bonds. This was demonstrated by the prominent vibrational increase in the frequency ranges of 1180-1300 cm⁻¹ (COC), 3200-3700 cm⁻¹ (OH stretching), 1600-1900 cm⁻¹ (CO) and the synchronous decrease in the C═O groups (bending) vibration intensities in the range of 1530-2500 cm⁻¹. The intensities of transmittance of aliphatic ester bonds present in PLGA were also decreased. Furthermore, crosslinks formed by the non-uniform length of polymeric chains resulted in a three-dimensional dense network that caused further vibrational intensities (FIG. 7).

2.8 Thermal Transitions within the PLGA Scaffolds

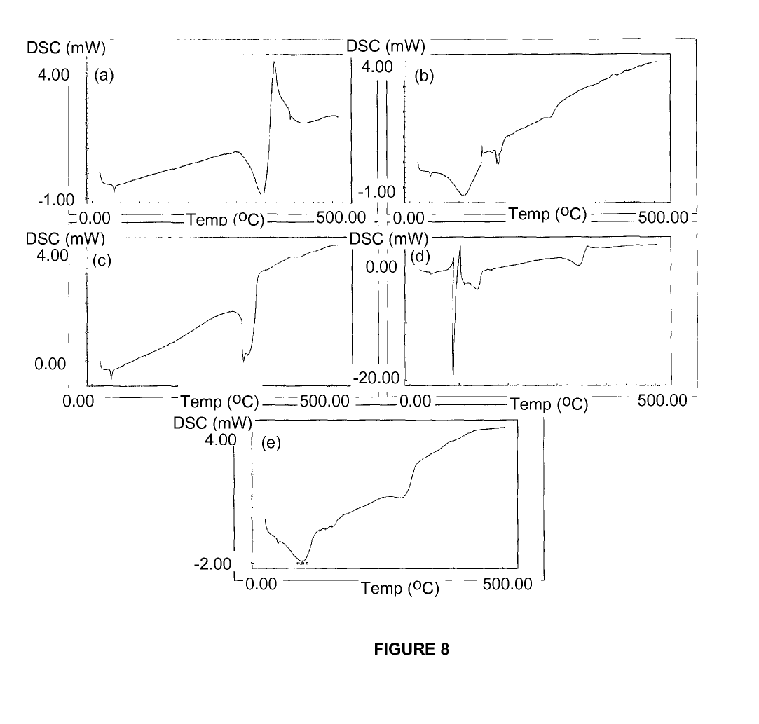

FIG. 8 demonstrates the enthalpy changes due to various polymer-salt interactions. During salting-out of PLGA, the enthalpy of the PLGA scaffolds was enhanced by the increase in steric strain attributable to a gain of electron energy. Furthermore, enthalpy changes also occurred as a result of bond formation that increased the bond-energy of the system and resonance stabilization thereby increasing the internal energy and enthalpy of the PLGA scaffolds. Furthermore, the steric strain caused by bond stretching, bond-angle deformation, and polymer-salt interactions increased the internal energy and enthalpy of the system. As molecules gained sufficient mobility to initiate the crosslinking reaction exothermic changes occurred in the temperature range of 40-47° C. which essentially described the glass transition point.

Table 4 lists the significant parameters obtained from analysis of the DSC profiles. In FIG. 8 a-e a step transition from glass to rubbery state on the heating cycle was clearly observed as sharp peaks at 47.24, 41.79, 40, 19, 43.35 and 42.82° C. respectively. The melting point range of PLGA scaffolds was 140-160° C. which was a significant reduction from native PLGA that has a melting point range of 280-300° C. Re-crystallization and further decomposition of the polymeric-salt complex took place between 410-430° C.

TABLE 4
Thermal parameters of native and
salted-out PLGA employing DSC

| Native PLGA | Tg° C. | mp° C. | Tc° C. | Td° C. |
|---|---|---|---|---|
| B | 47.24 | 280-300 | 354.85 | 411.65 |
| C | 41.79 | 148.30 | 285.61 | 428.05 |
| D | 40.19 | 280 | 315.30 | 426.94 |
| | 43.35 | 166.54 | 343.04 | 420.06 |

Tg = glass transition temperature; mp = melting point; Tc = re-crystallization temperature; Td = degradation temperature

Depending on the type of salt employed, water can be trapped within the polymeric matrix and thus depress the T_{g}. The size of ions (Al³⁺<Na⁺<Ca²⁺) determined the degree of T_{g} depression. Kelly and co-workers (1987) reported that a significant change in T_{g} is observed with a 10% increase or decrease in the water content within the polymeric matrix. Thus the dynamic activity of PLGA chains may be restricted by confines of water molecules within the matrix. The large ionic radius of Ca²⁺ led to an increase in the number of voids within the scaffold utilized by water molecules. Conversely, Na⁺ and Al³⁺ ions decreased the number of voids. Hence PLGA scaffolds salted-out with CaCl₂ had a lower T_{g} of 40.19° C. Studies by Paulaitis and co-workers (2004) have yielded verification on the dependence of polymeric hydration free energy on the solute size and shape. Moreover, work done by Bernazzani and co-workers (2003) demonstrated that precipitation of polymers from dilute solutions would depress the T_{g} which may be attributed to the crosslink induced shorter and free chain ends that disarray the crystallinity of the matrix. This was consistent in the findings of this study as well.

2.9 Characterization of the In Vitro Drug Release from the Monolithic Matrices

Theoretically, the primary drug release mechanism from both PLGA monolithic matrices should be diffusion through the matrix layer by a Fickian release mechanism. However, matrix swelling and erosion also played a significant role. Since melatonin is water-soluble and PLGA is a hydrophobic polymer, the rate of drug release decreased as a function of time as the diffusional path length for drug release increased over time when the dissolution medium front approached the center of the matrices.

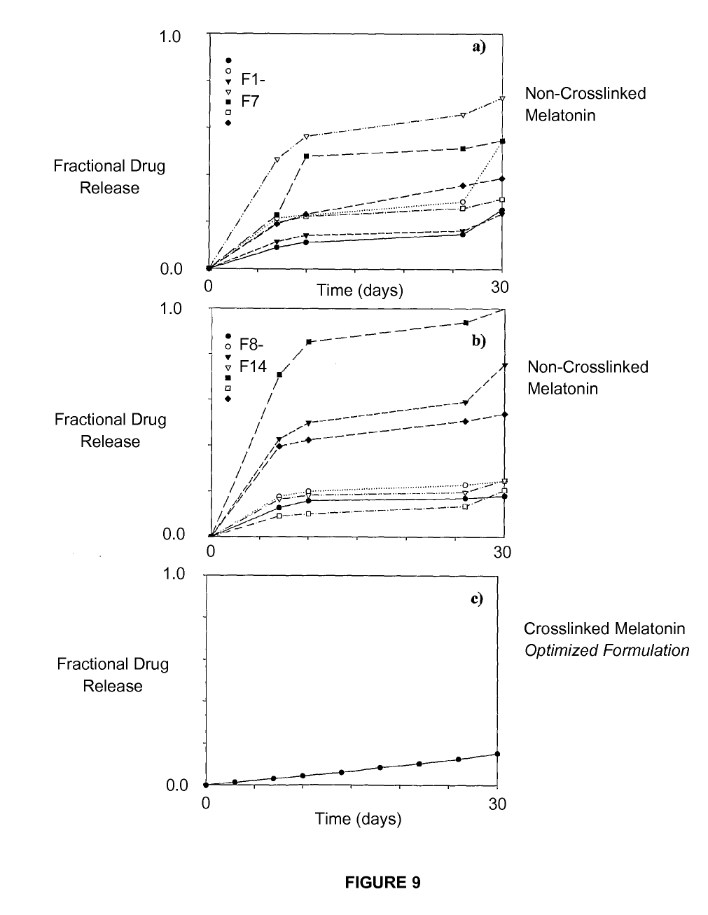

When melatonin was incorporated in a non-crosslinked manner, DEE varied between 46-90%. On the other hand, when melatonin was involved in the crosslinking process, an average DEE of 90% was achieved. Release profiles revealed that the monolithic matrices prepared by salting-out and subsequently crosslinking PLGA with melatonin employing various salts were able to achieve zero-order release kinetics with less than 20% melatonin released over a period of 30 days (FIG. 9 c). Monolithic matrices demonstrated a mean dissolution time at 30 days (MDT₃₀) of 6 to 26 (FIGS. 9 a and b). The fractional drug release (Mₜ/M_{∞}), and the drug release kinetics were calculated using the power taw Mₜ/M_{∞}=k₀t, where k, the kinetic constant was found to be k₀ of 0.004 to 0.038. The optimized formulation demonstrated 30-day zero-order kinetics for in vitro melatonin release (FIG. 9 c). This study demonstrated that crosslinking significantly controlled the rate of drug release due to the strong interactions between the drug and polymeric chains. The slow diffusion of melatonin from salted-out PLGA occurred due to shielding of polymeric reaction sites and coiling which prevented the maintenance of the same effective collision rate at which chemical reactions are Obtained between the same functional groups and the aqueous environment in non crosslinked polymer molecules, thus conferring the ability to achieve ideal zero-order drug release.

### 3. Conclusion

The salting-out and subsequent crosslinking approaches applied in the study in order to modify the physicochemical and the physicomechanical properties of native PLGA and achieve more controlled drug release kinetics displayed significant potential. The resulting crosslinked PLGA scaffolds exhibited superior structural integrity as determined by parameters such as resilience, energy of absorption and deformability moduli which contributed to the overall robustness and level of porosity of the PLGA scaffolds making it a favourable candidate for controlled drug delivery. The close correlation between the experimental and fitted response values demonstrated the reliability of the selected statistical design for experimental optimisation. The monovalent, divalent and trivalent ionic salts employed in the study proved to be suitable in transforming the structure of native PLGA into a modified PLGA scaffold with superior physicomechanical properties. These superior properties were confirmed by textural profile analysis, SEM, DSC and FTIR studies. In general, the degree of bond formation in the PLGA backbone demonstrated by vibrational intensity transitions from FTIR studies, in combination with the newly formed hydrolytically degradable PLGA crosslinks present a possible application of the PLGA scaffolds in rate-modulated drug delivery. This study has also demonstrated that salting-out and subsequent crosslinking of PLGA can significantly control the rate of drug release as a result of strong bonds formed between the drug and PLGA during crosslinking ultimately leading to zero-order release kinetics.

### Example B

### 4. Materials and Methods

### 4.1. Materials

Resomer® grades comprising PLGA with a 50% lactide content and 50% glycolide content and inherent viscosities ranging from 0.16 to 8.2 dl/g were utilized (Boehringer Ingelheim, Ingelheim, Germany). A water miscible solvent, typically a water miscible organic solvent, in this case N,N dimethyl formamide (DMF) was used as the solvent (Rochelle Chemical, Johannesburg, South Africa) and disodium hydrogen orthophosphate, sodium chloride and potassium dihydrogen phosphate were used to prepare the PBS (Saarchem (Pty) Ltd. Brakpan, South Africa). All other reagents were of analytical grade and used as supplied.

### 4.2. Formulation of the Salted-Out PLGA Scaffold

PLGA of various molecular masses designated as 1, 2 and 3 in Table 5 were weighed, dissolved in DMF, and placed in 200 mL glass beakers. Varying quantities of protonated water (pH 1.5) (H30+) was added to the polymeric solution to facilitate the induction of salting-out into scaffolds that were vacuum dried to remove excess solvent. The dehydrated scaffold samples were then immersed in 100 mL PBS (pH 7.4, 37 C) and oscillated at 100 rpm in a shaker bath (Stuart LABEX SBS4O). At 0, 7, 10, 26 and 30 days post-incubation the scaffolds were assessed for their physico-mechanical properties.

### 4.3. Construction of the Experimental Design

Table 5 lists the normalized factor levels for the independent formulation variables. A Face-Centered Central Composite Design (FCCD) was selected for optimization of the PLGA scaffolds. The statistical model allows for simultaneously studying the effect of several independent formulation variables influencing the desired responses, by altering the variables in a limited number of experiments. FCCD was employed in this study to determine coefficients of a second order. This is more superior to conventional methods of optimization which involves varying one factor at a time, while keeping constant all other parameters, thus not screening the main interactions and effects of all the involved factors simultaneously (Table 6).

TABLE 5

| Independent | Factor level | | | |
|---|---|---|---|---|
| variables | Low | Medium | High | Units |
| Water volume | 10 | 55 | 100 | mL |
| PLGA molecular | 1 | 2 | 3 | Da |
| mass a | | | | |
| PLGA | 1 | 5.5 | 10 | % w/v |
| concentration | | | | |
| Salting-out | 2 | 13 | 24 | h |
| reaction time | | | | |

a PLGA molecular mass: 1 = 55,000 Da, 2 = 100,000 Da, 3 = 160,000 Da.

TABLE 6
Randomized experimental runs generated from the FCCD

| Formulation | Water | | | Salting-out |
|---|---|---|---|---|
| No. | volume | PLGA Mw -a | [PLGA] b | reaction time |
| I | 10 | I | 10 | 2 |
| 2 | 100 | 2 | 5.5 | 13 |
| 3 | 100 | I | I | 24 |
| 4 | 10 | 3 | I | 24 |
| 5 | 10 | I | I | 2 |
| 6 | 55 | 2 | I | 13 |
| 7 | 100 | I | I | 2 |
| 8 | 55 | 2 | 10 | 13 |
| 9 | 10 | 2 | 5.5 | 13 |
| 10 | 10 | 3 | 10 | 24 |
| II | 55 | 2 | 5.5 | 13 |
| 12 | 100 | 3 | 10 | 24 |
| 13 | 55 | 2 | 5.5 | 13 |
| 14 | 100 | I | 10 | 2 |
| 15 | 55 | 3 | 5.5 | 13 |
| 16 | 55 | I | 5.5 | 13 |
| 17 | 55 | 2 | 5.5 | 2 |
| 18 | 100 | 3 | I | 2 |
| 19 | 10 | 3 | 10 | 2 |
| 20 | 10 | I | 10 | 24 |
| 21 | 10 | I | I | 24 |
| 22 | 100 | I | 10 | 24 |
| 23 | 100 | 3 | 10 | 2 |
| 24 | 55 | 2 | 5.5 | 24 |
| 25 | 10 | 3 | I | 2 |
| 26 | 100 | 3 | I | 24 |

a- PLGA molecular mass: 1 = 55,000 Da, 2 = 100,000 Da, 3 = 116,000 Da.
b PLGA concentration

### 5. Results and Discussion

### 5.1. Stereomicroscopic Image of a Salted-Out PLGA Scaffold

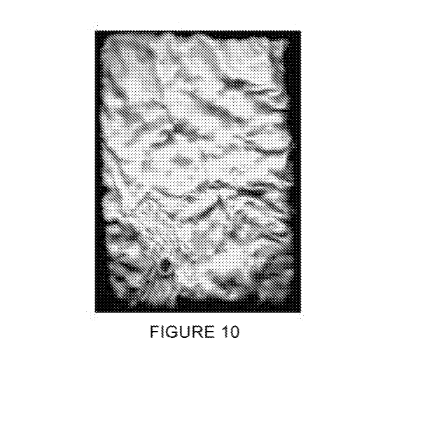

FIG. 10 depicts the surface morphology of a woven composite flat-shaped salted-out PLGA scaffold (thickness=1 mm) obtained. Surface folds and inter-connectivity, resembling the body's extra-cellular matrix were present. The exterior roughness and interconnected folds may be ascribed to crosslinked fibers within the PLGA matrix to form a scaffold. These folds provide an increased surface area for further drug entrapment as well as cell-seeding in drug delivery and tissue engineering.

5.2. Simulation of PLGA Monomeric Interactions During Solubilization, Salting-Out and Subsequent Crosslinking into the Scaffold

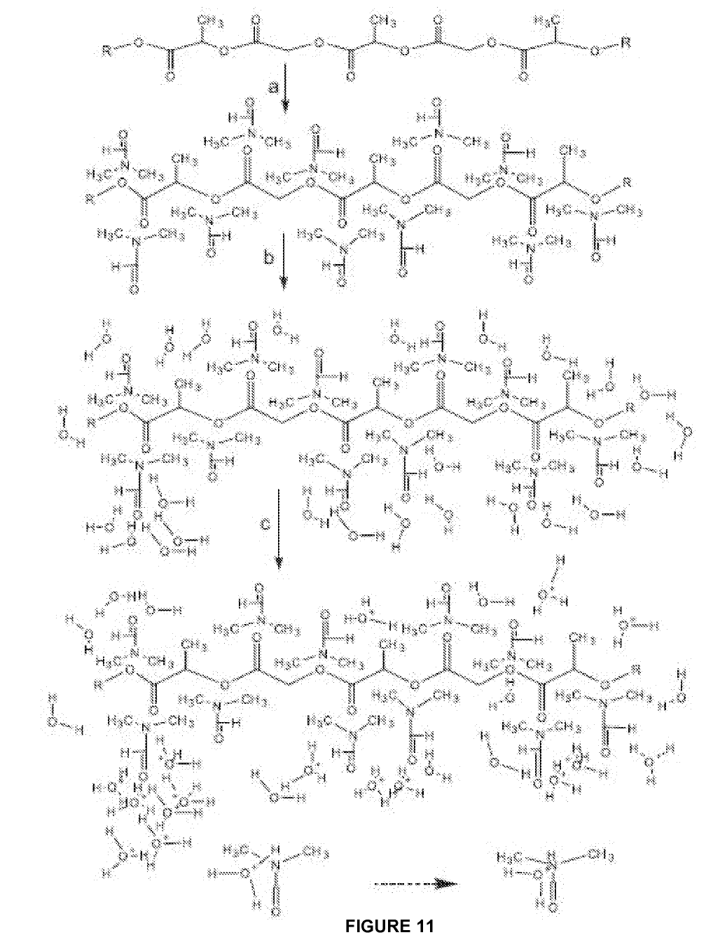

Solvation was conducted via mixed quantum/molecular mechanics. This allowed for computing the solvation energy and the solvent effects at a molecular interaction level, The interactions between PLGA, DMF, H₂O and HCI are depicted in FIG. 11. The model predicted experimentally observed trends such that by increasing the pH of the system resulted in increased partitioning of PLGA that led to immediate salting-out and subsequent crosslinking of native PLGA. Crosslinking of the matrix occurred mainly by covalent bonding of PLGA molecules and crosslinking ions to form a three-dimensional scaffold.

During solubilization of PLGA in DMF, two distinct stages were observed. Initially PLGA adsorbs DMF to form a gel-like stage that slowly dispersed into solution. During the second stage, PLGA chains assumed a configuration such that the attractive and repulsive forces on each chain were precisely counter-balanced. The coupled effect of these forces induced the PLGA chain to adopt a meticulous stereochemical configuration, with a free rotation about the chiral carbons that resulted in the unperturbed formation of the C—C—C bond-angle fixed at 109° and the C—C molecular distance of 1.54 A, as depicted in FIG. 11. Accordingly, this model allowed one to hypothesize the relative importance of polymer-solvent (PLGA-DMF) and ion-solvent (H+-DMF) interactions on phase separation during salting-out of native PLGA to form the scaffold.

FIG. 11(a) depicts the ionic bond interactions between the N—O atoms of DMF and PLGA, respectively. In FIG. 11(b), due to the hydrophobicity of PLGA water clathrates formed at the surfaces that resulted in an outer solvation shell. In FIG. 11(c), as the reaction proceeded ionic interactions involving H₂O and DMF occurred. The salting-out effect increased due to acidity of the system and thus the polar aprotic solvent was key during the reaction. Furthermore, the salting-out of PLGA into scaffolds may have also been enhanced by the ‘Hydrophobic Effect’. Owing to the hydrophobicity of PLGA, junction zones formed around the PLGA, which affected the configuration and solvation shells of H₂O, directing them away from the immediate surface of the PLGA. The enthalpy changes during salting-out and crosslinking of PLGA resulted from bond formation, resonance, and steric strain. Bond stretching, bond-angle deformation, as well as interactions between atoms augmented the internal energy of the system.

Formation of a PLGA scaffold decreased chain delocalization and configurational changes of the structure during salting-out brought about transitions in the physicochemical and physicomechanical properties of native PLGA. However, these properties are anticipated to transform as the scaffolds erode in PBS over time. In order to assess the integrity of the salted-out PLGA scaffold during residence in PBS the physicomechanical transitions of PLGA scaffolds were assessed.

### 5.3. Physicomechanical Analysis of the PLGA Scaffolds Employing Textural Profile Analysis

The physicomechanical transitions of the PLGA scaffolds interacting with the PBS environment were analyzed. The extent of physicomechanical modification was quantified in order to predict the dynamic transitions for flexibility and reproducibility of each scaffold variant. The PLGA scaffolds degraded at varying rates when immersed in PBS. Scaffolds interacted with the PBS by means of polarhydrophobic interactions and hydrogen bonding. The physicomechanical properties of the scaffolds were governed by the PLGA molecular mass, PLGA concentration, volume of water and salting-out reaction time. As the crosslinking density is reduced, the matrices had an increased resilience and a decreased degradation rate in PBS.

The ability of the scaffold to absorb energy was found to correlate with the residence time in PBS. Higher water volumes caused a decreased quantity of energy to be absorbed by the scaffolds during textural probe penetration. On the contrary, higher PLGA molecular masses caused an increased resistance to textural probe penetration. This indicated that excess energy was absorbed by the scaffolds when higher molecular masses of PLGA were used. The peak energy absorbed by the scaffolds occurred at day 10. The mass variation of scaffolds was also observed to decrease with an increase in scaffold residence in PBS, which signified erosion. The general trend revealed by the data was that the scaffolds with higher PLGA molecular masses and concentrations were less susceptible to degradation and physicomechanical transitions when immersed in PBS. The salted-out PLGA scaffolds produced were more hydrophobic and hence less prone to hydrolysis in PBS, hence, retarding degradation and conferring further flexibility to control drug release.

### 5.4. Response Surface Optimization of Scaffold Formulations

By utilizing the salting-out and crosslinking process scaffolds with novel physicomechanical properties were obtained. Surface plots were able to focus on establishing the optimal combination of independent formulation variables for the enhancement of the scaffold physicomechanical properties making them suitable for controlled drug delivery.

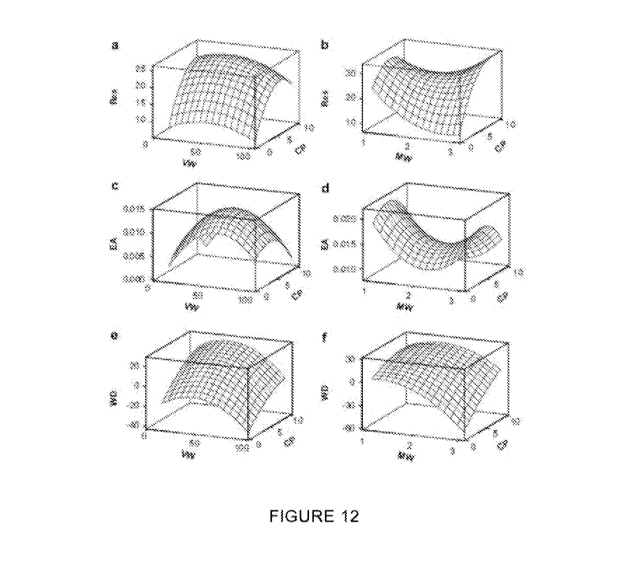

Response surface plots indicated that increasing the PLGA concentration increased MR in spite of changes in water volume (FIG. 12(a)). The PLGA molecular mass increased MR up to an optimal level of 100,000 Da. As the PLGA molecular mass increased further MR decreased linearly (FIG. 12(b)). Observations also highlighted that higher PLGA molecular masses increased MR only when the salting-out reaction time was longer. The salting-out reaction time proved to be effective in augmenting the PLGA concentration in increasing MR.

FIG. 12(c) demonstrated that water volume was found to influence the quantity of energy absorbed during textural probe penetration of the scaffolds. Higher water volumes caused lower quantities of energy to be absorbed. The increased volume of water during salting-out resulted in the entrapment of excess water molecules within the polymeric voids coupled with ion hydration, thereby decreasing the absorption of hydration-free energy. Higher PLGA concentrations and molecular mass resulted in increased energy absorption (FIG. 12(d)). FIG. 12(e) and (I) demonstrated similar trends for mass deflection as shown by FIGS. 12(a)-(d) and concluded a fairly close correlation between all three physicomechanical properties of the PLGA scaffolds formed. No statistically significant differences were noted between the fitted and experimental values (p>0.05).

### 5.5 Conclusion

A novel salted-out and crosslinked monolithic delivery dosage form has been prepared. The PLGA scaffolds prepared as described above can typically be compressed to form tablets.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention. Moreover, the present concepts expressly include any and all combinations and subcombinations of the preceeding elements and aspects.

What is claimed is:

1. A monolithic drug delivery dosage form comprising a salted-out and crosslinked polymer having a pharmaceutically active agent disposed therewith, wherein the polymer is poly-lactic co-glycolic acid and is crosslinked and salted-out with a crosslinking agent selected from the group consisting of: sodium chloride, aluminium chloride and calcium chloride, such that bonding occurs between the polymer and the crosslinking agent to form an independent crosslinked and salted-out product which entangles the pharmaceutically active agent, the monolithic drug delivery dosage form having a zero order release of the pharmaceutically active agent on contact with an aqueous medium.

2. The monolithic drug delivery dosage form as claimed in claim 1, wherein the monolithic drug delivery dosage form is compressed into a tablet.

3. The monolithic drug delivery dosage form as claimed in claim 1, wherein the zero order release of the pharmaceutically active agent lasts for a period of up to 30 days.

4. The monolithic drug delivery dosage form as claimed in claim 1, wherein the poly-lactic co-glycolic acid has a 1:1 lactide:glycolide ratio.

5. The monolithic drug delivery dosage form as claimed in claim 1, wherein the pharmaceutically active agent is melatonin.

6. The monolithic drug delivery dosage form as claimed in claim 2, wherein the pharmaceutically active agent is melatonin.

7. The monolithic drug delivery dosage form as claimed in claim 3, wherein the pharmaceutically active agent is melatonin.

8. The monolithic drug delivery dosage form as claimed in claim 4, wherein the pharmaceutically active agent is melatonin.

9. A method of producing a monolithic drug delivery dosage form comprising a pharmaceutically active agent characterised in that the method includes the steps of salting-out and crosslinking poly-lactic co-glycolic acid with a crosslinking agent selected from the group consisting of: sodium chloride, aluminium chloride and calcium chloride.

10. The method as claimed in claim 9, wherein the steps of salting-out and crosslinking poly-lactic co-glycolic acid with a crosslinking agent includes the follow steps:
(a) dissolving poly-lactic co-glycolic acid in a water miscible solvent to form a polymeric solution;
(b) adding the pharmaceutically active agent to the polymeric solution;
(c) adding a crosslinking agent selected from the group consisting of: sodium chloride, aluminium chloride and calcium chloride, so as to entangle the pharmaceutically active agent with the poly-lactic co-glycolic acid; and
(d) salting-out the crosslinked poly-lactic co-glycolic acid of step (c) to form a monolithic drug delivery dosage form having zero order release of the active pharmaceutical agent on contact with an aqueous medium.

11. The method of claim 10, further comprising an additional step, Step (e), comprising compressing the crosslinked and salted-out poly-lactic co-glycolic acid to form a tablet

12. The method as claimed in claim 9, wherein the poly-lactic co-glycolic acid has a 1:1 lactide:glycolide ratio.

13. The method as claimed in claim 10, wherein the water miscible solvent is at least one selected from the group comprising: acetone and N,N-dimethyl formamide (DMF).

14. The method as claimed in claim 9, wherein said pharmaceutically active agent is water-soluble.

15. The method as claimed in claim 10, wherein said pharmaceutically active agent is melatonin.

16. The method as claimed in claim 11, wherein said pharmaceutically active agent is melatonin.

17. The method as claimed in claim 12, wherein said pharmaceutically active agent is melatonin.

18. The method as claimed in claim 13, wherein said pharmaceutically active agent is melatonin.

19. The method as claimed in claim 9, wherein step (d) comprises the addition of H3O+.
